(19) United States
(12) Patent Application Publication
(10) Pub. No.: US 2010/0193386 A1
(43) Pub. Date: Aug. 5, 2010

(54) Vaginal-Health Products Comprising Colored Applicators

(76) Inventors: Adrienne Rae Loyd, Neenah, WI (US); Andrew Pottle Meurer, Neenah, WI (US); Christopher Patrick Worzalla, Hortonville, WI (US); Kristin Ann Goerg-Wood, Sherwood, WI (US); Amy Lynn Attenberger, Kimberly, WI (US); Jennifer Lea Westemeyer, Neenah, WI (US); Sarah Jane Marie Freiburger, Hortonville, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/645,920

(22) Filed: Dec. 23, 2009

Related U.S. Application Data
(60) Provisional application No. 61/206,459, filed on Jan. 30, 2009.

(51) Int. Cl. A61F 13/32

(52) U.S. Cl. 206/438; 604/15

(57) ABSTRACT

A package containing vaginal-health products comprises a first vaginal-health product comprising a first applicator. The first applicator comprises a first barrel portion, a first plunger portion, and a first pledget at least partly contained in the first barrel portion, where the first barrel portion has a first barrel color. The package also contains a second vaginal-health product comprising a second applicator. The second applicator comprises a second barrel portion, a second plunger portion, and a second pledget at least partly contained in the second barrel portion, where the second barrel portion comprises a second barrel color. In addition, the first barrel color is different from the second barrel color, and the first pledget is the same as the second pledget.

### PRIORITY

This non-provisional application claims priority of Provisional Application No. 61/206,459, filed on Jan. 30, 2009. The entirety of Application No. 61/206,459 is incorporated herein by reference.

### BACKGROUND

The way women think about feminine-hygiene products is shaped by many factors, including the culture, the media, family, peers, view of self, and the history of these products in the country of purchase. Even the phrase “feminine-hygiene product,” and words that describe these products and their use, such as “cycle,” or “protection,” present a story or narrative.

These factors, and the narrative or story about these products as presented by consumer products manufacturers, often result in people making an association between feminine-hygiene products, such as tampons, and a clinical or medical condition. As a result, young women may view menstruation and vaginal health as medical in nature, perhaps something that is not normal, rather than a natural part of who they are. This association between feminine-hygiene products and a medical condition may so shape a user's perception of these products that she may be reluctant to talk openly about her body, menstruation, and vaginal health. This is unfortunate. The more a young woman understands herself, and what is going on with her body, the better.

Alternatively, this association between feminine-hygiene products and a medical condition may conflict with how a young, modern woman views herself and her body. If the young woman has moved beyond taboos associated with vaginal health, and instead perceives herself as open and in command, and that vaginal health, including menstruation, is merely a part of who she is, then this historic linkage with a medical condition may be viewed as outdated and irrelevant. And if a manufacturer's choice of words, images, and color expressly or implicitly links these products to what is perceived as a clinical or medical condition, then these same young women may reject the manufacturer's products, or may continue to believe that menstruation is clinical or medical in nature.

For many years feminine-hygiene products, including tampons, were typically white. Many people, of course, associate the color white with clinics, hospitals, and medical care, which can reinforce the association and the cultural taboos mentioned above.

In some cases, manufacturers have made and sold printed feminine-care pads, and colored wrappers containing individual feminine-care products, to promote some aspect of femininity. When color is used, all products in a given package typically have the same color or print pattern. In an effort to make feminine hygiene products look more “feminine,” many manufacturers have employed, for example, a pastel pink color, or floral patterns. Many modern women, especially young women, may not view these colors and images as being feminine, or even desirable. Instead, they may view these colors and images as reflecting predictable and stereotypical choices. Women in today's society are dynamic and have multi-faceted personalities. They pay attention to detail and like to express themselves in ways that reflect their unique personalities and moods. This can be seen for instance in the fashion industry, where popular fashions incorporate a mix of designs, colors, shapes, and patterns and often incorporate an element of uniqueness and risk. Women choose combinations from this fashion mix that reflect their personality. For this reason, many women may not view stereotypical feminine cues, such as those given above, as “feminine.”

Manufacturers have also tied color to product characteristics, so that color conveys information to a user about the product. Thus, for example, one manufacturer has tied the color of plastic tampon tube applicators to the absorbency of the absorbent pledget contained in the applicator. If a woman is using a regular-absorbency applicator tampon, then she must use the single color associated with regular absorbency. While this approach is in some ways useful, the manufacturer is constrained to using a color—the color used to convey information about absorbency—for a given product form. In other words, when a woman buys regular-absorbency applicator tampons in a package, all of the applicators contained inside are of the same color (i.e., the color selected by the manufacturer, not the user, to signify regular absorbency).

Thus, there is a need to provide to women the ability to choose different colored vaginal-health products, such as tampon products, from a single package that: (1) is consistent with, and helps reinforce, the fact that a woman is in command of her life and her body; (2) is surprising in a category where the color white is often employed; (3) helps overcome cultural taboos about vaginal health; (4) helps overcome the perception that menstruation represents a medical condition, rather than a natural occurrence in a healthy body; and/or (5) provides a fashion mix that reflects a woman's personality, thus giving a woman the ability to choose a color she feels like using on a particular occasion.

### SUMMARY

Generally, a woman inserts a tampon applicator containing an absorbent pledget into her body; deploys the absorbent pledget into her vaginal canal, typically by depressing a plunger portion of the applicator; and then withdraws the applicator, leaving the absorbent pledget in her body to absorb menstrual fluid. The same can apply in the case of a vaginal pessary in which the pledget may or may not be absorbent. In response to the needs discussed above, we have found that women are surprised, and delighted, to have the choice of different colored applicators, contained in a single package, for use in deploying the pledget, such as an absorbent pledget, contained in the applicator. This of course means that color is not used solely to convey information about absorbency, as is the case with some current tampon products. Instead, other independent indicia of absorbency may be provided, thereby allowing the manufacturer to give women the choice of different colored applicators in a single package.

Applicator tampons are typically wrapped individually. For a user to pick a desired applicator color from a number of different applicator colors contained in a single package, either some portion of the wrapper is transparent so that the user can see the applicator directly; or the wrapper itself has some indicia of the color of the applicator (e.g., a tampon applicator) inside the wrapper. Thus, in some aspects, the color of the wrapper, or some portion thereof, can be substantially the same as the color of the applicator inside the wrapper.

The package containing different colored applicators may have text, images, or other indicia disposed on or in the package relating one or more applicator colors to, for example, a state of being of the user. If one of the applicators is the color yellow, then indicia may relate yellow, for example, to a positive attitude, or feeling upbeat. This indicia may be employed on the package, on the wrappers around each of the applicator products contained in the package, on the applicators themselves, or some combination thereof.

In one exemplary version, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. Typically the first plunger portion is slidably positioned, at least in part, inside the first barrel portion. A first pledget, such as an absorbent pledget or a pessary device, is at least partly contained in the first barrel portion. Generally, a user pushes on the first plunger portion, thereby causing the first pledget to exit through the end of the first barrel portion, with the pledget remaining in the user's vaginal canal after the first applicator is withdrawn. The package also includes a second vaginal-health product. The second vaginal-health product has a second applicator. The second applicator has both a second barrel portion and a second plunger portion, with the second barrel portion having a second barrel color. The first barrel color is different from the second barrel color. And the first pledget is the same as the second pledget.

Another exemplary version of the invention includes features described in the previous paragraph. Furthermore, the first plunger portion comprises a first plunger color, and the second plunger portion comprises a second plunger color, with the first plunger color being the same as the second plunger color. In a variation of this embodiment, the package includes instructions on using the vaginal-health product that refer to the color of the plunger. For example, if both the first and second plunger portions are blue, the instructions on the package might state that a user is to depress that portion of the applicator that is colored blue. Of course other approaches may be used to communicate instructions, either alone or in combination with text (e.g., a simple colored graphic in which the plunger portion is colored blue).

In another exemplary version, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. The first plunger portion comprises a first plunger color that is substantially the same as the first barrel color. A first pledget, such as an absorbent pledget or a pessary device, is at least partly contained in the first barrel portion. The package also includes a second vaginal-health product. The second vaginal-health product has a second applicator. The second applicator has both a second barrel portion and a second plunger portion, with the second barrel portion having a second barrel color. The second plunger portion comprises a second plunger color that is substantially the same as the second barrel color. The first barrel color is different from the second barrel color. The first pledget is the same as the second pledget.

In another version of the invention, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. The first plunger portion comprises a first plunger color that is different from the first barrel color. A first pledget, such as an absorbent pledget or a pessary device, is at least partly contained in the first barrel portion. The package also includes a second vaginal-health product. The second vaginal-health product has a second applicator. The second applicator has both a second barrel portion and a second plunger portion, with the second barrel portion having a second barrel color. The second plunger portion comprises a second plunger color that is different from the second barrel color. The first barrel color is different from the second barrel color. The first pledget is the same as the second pledget.

In another exemplary embodiment, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. The first plunger portion comprises a first plunger color that is a different shade of the first barrel color. A first pledget, such as an absorbent pledget or a pessary device, is at least partly contained in the first barrel portion. The package also includes a second vaginal-health product. The second vaginal-health product has a second applicator. The second applicator has both a second barrel portion and a second plunger portion, with the second barrel portion having a second barrel color. The second plunger portion comprises a second plunger color that is a different shade of the second barrel color. The first barrel color is different from the second barrel color. The first pledget is the same as the second pledget.

In other exemplary versions of the invention, any of the embodiments described above are such that the first vaginal-health product is contained in a first wrapper comprising a first wrapper color that is substantially the same as the first barrel color, and the second vaginal-health product is contained in a second wrapper comprising a second wrapper color that is substantially the same as the second barrel color.

In other versions of the invention, packages like those described in the preceding paragraphs are such that the first vaginal-health product is contained in a first wrapper comprising a window through which a portion of the first vaginal-health product is viewable, and the second vaginal-health product is contained in a second wrapper comprising a window through which a portion of the second vaginal-health product is viewable.

In some exemplary versions of the invention, packages like those described above are such that the first and second pledget are each an absorbent pledget having an absorbency level (e.g., each absorbent pledget having a “regular” absorbency).

In another exemplary version, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. A first pledget in the form of an absorbent pledget having a particular absorbency level is at least partly contained in the first barrel portion. The first vaginal-health product is contained in a first wrapper on which is disposed indicia of the absorbent pledget's absorbency level, said indicia being unrelated to the first barrel color. The package also includes a second vaginal-health product. The second vaginal-health product has a second applicator. The second applicator has both a second barrel portion and a second plunger portion, with the second barrel portion having a second barrel color. The first barrel color is different from the second barrel color. The first and second plunger colors may be the same as the corresponding first and second barrel colors, or they may be different. The second pledget is also in the form of an absorbent pledget having an absorbency level the same as the first pledget (e.g., each absorbent pledget having a “regular” absorbency). The second vaginal-health product is contained in a second wrapper on which is disposed indicia of the absorbency level, said indicia being unrelated to the second barrel color.

In other versions of the invention, packages having features described in the preceding paragraphs have a viewable surface area, and at least 30% of the viewable surface area is black. If the package has the general shape of a rectangular parallelepiped (i.e., 4 rectangular side panels, a rectangular top panel, and a rectangular bottom panel), then “viewable surface area” means the sum of the six areas corresponding to each of the six viewable rectangles or panels that make up the rectangular parallelepiped. We have found that women are delighted, and surprised, to see multiple colors contained in a package that is starkly different from packages that have been offered historically in the feminine-care category. Accordingly, other versions of the invention have even higher percentages of the viewable surface area as black: 50%, 75%, or even 90%, or more.

In another exemplary embodiment, packaging having features described in the preceding paragraphs comprise a window through which some portion of the contents of the package are viewable. Typically the wrappers that contain the vaginal-health products are seen through the package window, rather than the vaginal-health products contained in the wrappers. Of course if the wrapper comprises a window, or some other portion through which the vaginal-health product is viewable through the wrapper, then the vaginal-health product itself may be seen through the package window if the wrapper window or portion is viewable through the package window.

Another version of the invention is an array of vaginal-health products. The array comprises a first package and a second package, each containing vaginal-health products. The first package comprises a first vaginal-health product and a second vaginal-health product. The first vaginal-health product comprises a first applicator, the first applicator having a first barrel portion and a first plunger portion. The first barrel portion comprises a first barrel color. The first vaginal-health product also comprises a first pledget that is at least partly contained in the first barrel portion. The second vaginal-health product comprises a second applicator, the second applicator having a second barrel portion and a second plunger portion. The second barrel portion comprises a second barrel color. The second vaginal-health product also comprises a second pledget that is at least partly contained in the second barrel portion. The first barrel color is different from the second barrel color, and the first pledget is the same as the second pledget (e.g., both the first and second pledgets are absorbent pledgets having a regular absorbency). The array also comprises a second package. The second package contains vaginal-health products comprising applicators, with the applicators in the second package each having the same appearance (e.g., the same color).

Another version of the invention is an array like that described in the preceding paragraph. The first package has a first viewable surface area, and at least 30% of the first viewable surface area is black. The second package has a second viewable surface area, and at least 30% of the second viewable surface area is white. The packages may be made of plastic film, paperboard, or some combination of these. In one version of the invention, the first package is composed of paperboard, and the second package is composed of plastic film. The packages may also incorporate a window through which the contents of the packages are viewable. In another version of the invention, the first package comprises a window through which the contents of the first package are viewable. The second package does not comprise a window.

In another version of the invention, the package has features like those described generally in the preceding paragraphs. In some aspects, the invention is a package containing vaginal-health products. The package includes a first vaginal-health product and a second vaginal-health product. The first vaginal-health product has a first applicator. The first applicator has both a first barrel portion and a first plunger portion, with the first barrel portion having a first barrel color. A first pledget, such as an absorbent pledget or a pessary device, is at least partly contained in the first barrel portion. The package also includes a second vaginal-health product. The second vaginal-health product has a second applicator. The second applicator has both a second barrel portion and a second plunger portion, with the second barrel portion having a second barrel color. The first barrel color is different from the second barrel color. And the first pledget is the same as the second pledget. The package also comprises indicia relating the first barrel color to a state of being of the user. Furthermore, the package may comprise indicia for a user's ability to choose different colors.

The exemplary versions above refer to applicators comprising at least a barrel portion and a plunger portion. Other representative versions of the invention include applicators having the features recited above, and further comprising additional components, such as a telescoping portion that interconnects the plunger portion and the barrel portion, for example. This exemplary embodiment is described in more detail below. The additional components may have a color that is: (1) the same or different than the barrel portion, or (2) the same or different than the plunger portion. Furthermore, any of the additional components may be transparent, translucent, or opaque (as may be the other portions; and, of course, whether transparent, translucent, or opaque, color may be added as needed to achieve the features described above; e.g., if the plunger is constructed of a clear plastic, and it was to be colored, the clear plastic could be tinted).

For embodiments of the present invention in which a package contains wrappers having different colors, the wrapped vaginal-health products may be randomly placed in the package. Alternatively, the wrapped vaginal-health products may be placed in the package according to a pre-selected arrangement.

One version of the invention is a method of selecting a vaginal-health product comprising a colored applicator. The method comprises the steps of: (i) positioning in the hands of a user a plurality of vaginal-health products having different applicator colors; and (ii) providing instructions that a user may select a vaginal-health product on the basis of color.

In some particular aspects, a package containing vaginal-health products comprises a first vaginal-health product comprising a first applicator, the first applicator comprising a first barrel portion, a first plunger portion and a first pledget at least partly contained in the first barrel portion, where the first barrel portion comprises a first barrel color. The package also contains a second vaginal-health product comprising a second applicator, the second applicator comprising a second barrel portion, a second plunger portion and a second pledget at least partly contained in the second barrel portion, where the second barrel portion comprises a second barrel color. The first barrel color is different from the second barrel color, and the first pledget is the same as the second pledget. In further aspects, the first plunger portion comprises a first plunger color, the second plunger portion comprises a second plunger color, and the first plunger color is the same as the second plunger color. In addition, instructions on use of the vaginal-health product that reference the color of the plunger can be disposed on the package. In yet further aspects, the first plunger portion comprises a first plunger color that is substantially the same as the first barrel color, and the second plunger portion comprises a second plunger color that is substantially the same as the second barrel color. In still further aspects, the first plunger portion comprises a first plunger color that is different from the first barrel color, and the second plunger portion comprises a second plunger color that is different from the second barrel color. In yet further aspects, the first plunger portion comprises a first plunger color that is a different shade of the first barrel color, and the second plunger portion comprises a second plunger color that is a different shade of the second barrel color. In still further aspects, the first vaginal-health product is contained in a first wrapper comprising a first wrapper color that is substantially the same as the first barrel color, and the second vaginal-health product is contained in a second wrapper comprising a second wrapper color that is substantially the same as the second barrel color to provide wrapped vaginal-health products. In yet further aspects, the arrangement of vaginal-health products inside the package comprise a pattern. In still further aspects, the first vaginal-health product is contained in a first wrapper comprising a window through which a portion of the first vaginal-health product is viewable, and the second vaginal-health product is contained in a second wrapper comprising a window through which a portion of the second vaginal-health product is viewable. In yet further aspects, the first pledget and the second pledget are each an absorbent pledget having the same absorbency level. In addition, the first vaginal-health product can be contained in a first wrapper comprising a first wrapper color that is substantially the same as the first barrel color; the second vaginal-health product can be contained in a second wrapper comprising a second wrapper color that is substantially the same as the second barrel color; where the first wrapper can further comprise a first indicia of the absorbency level, the first indicia being unrelated to the first barrel color; and where the second wrapper can further comprise a second indicia of the same absorbency level, the second indicia being unrelated to the second barrel color. In still further aspects, the package has a viewable surface area, wherein at least 30% of the viewable surface area is black. In addition, the package can further comprise a window through which some portion of the contents of the package is viewable. In yet further aspects, the package further comprises indicia relating at least the first barrel color to a state of being of a user. In still further aspects, the package further comprises indicia relating to a user's ability to choose different colors.

In some particular aspects, an array of packages of vaginal-health products comprises a first package containing vaginal-health products, the first package comprising a first vaginal-health product comprising a first applicator, the first applicator comprising a first barrel portion and a first plunger portion, wherein the first barrel portion comprises a first barrel color; and a first pledget at least partly contained in the first barrel portion; and a second vaginal-health product comprising a second applicator, the second applicator comprising a second barrel portion and a second plunger portion, wherein the second barrel portion comprises a second barrel color; and a second pledget at least partly contained in the second barrel portion; wherein the first barrel color is different from the second barrel color; and wherein the first pledget is substantially the same as the second pledget. The array further comprises a second package containing vaginal-health products comprising applicators, wherein the applicators in the second package all have the same appearance. In addition, the first package and the second package have indicia of the same brand. In further aspects, the first package has a first viewable surface area and at least 30% of the first viewable surface area is black, and the second package has a second viewable surface area where at least 30% of the second viewable surface area is white. In still further aspects, the first package is composed of paperboard and the second package is composed of plastic film. In addition, at least the first package can comprise a window.

In some particular aspects, a package containing vaginal-health products comprises a first vaginal-health product comprising a first applicator having a first applicator color, where the first applicator is disposed within a first wrapper having a first wrapper color; and a second vaginal-health product comprising a second applicator having a second applicator color, where the second applicator is disposed within a second wrapper having a second applicator color; where the first applicator color is the same as the second applicator color; and where the first wrapper color is different than the second wrapper color. In further aspects, the first wrapper comprises a first indicia having a first indicia color and the second wrapper comprises a second indicia having a second indicia color; where the first indicia color and the second indicia color are unrelated to the the first applicator color and the second applicator color; where the first indicia color is the same as the second indicia color; where the first indicia color and the second indicia color indicate the absorbency level of the first vaginal-health product and the second vaginal-health product; and where the first vaginal-health product and the second vaginal-health product have the same absorbency level. In still further aspects, the package further comprises a window through which some portion of the contents of the package are viewable. In yet further aspects, the package has a viewable surface area, where at least 30% of the viewable surface area is black.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1A shows an exemplary embodiment of a vaginal-health product of the present invention including an applicator comprising a barrel portion and a plunger portion.

FIG. 1B shows an exemplary embodiment of a vaginal-health product of the present invention including an applicator comprising a barrel portion, a plunger portion and an additional component.

FIG. 2A shows an exemplary embodiment of a package of the present invention having an optional indicia and an optional window.

FIG. 2B shows an exemplary embodiment of a package of the present invention having an optional indicia and an optional window and having vaginal-health products disposed therein.

FIG. 3 shows one version of a diagram used to characterize color.

FIG. 4 shows an exemplary embodiment of a wrapper of the present invention.

FIG. 5 shows an array of the present invention including packages having vaginal-care products disposed within.

Repeated use of reference characters in the present specification and drawings is intended to represent the same or analogous features or elements of the present invention. The drawings are representational and are not necessarily drawn to scale. Certain proportions thereof may be exaggerated, while others may be minimized

### TEST METHODS

Unless otherwise noted, all tests are performed at a temp of 23° C. and a relative humidity of 50%.

### Color Measurement Test

For the present application, hue, luminosity and saturation/vividness can be measured as described in the following paragraphs.

A Zeiss KS400 Image Analysis system is used for feature identification and colorimetric measurement. The Zeiss KS400 image analysis incorporates a Zeiss AxioCam color CCD camera (1300×1030 pixels, 3 channel color, 8 bit per channel) having a 20 mm AF-Nikkor lens (f/2.8). The camera is mounted vertically facing down onto a sample stage and has an effective field of view of 97 mm×80 mm. Incident sample stage illumination is effected by four incandescent floodlamps (Sylvania) on a double Variac (70%; 90%), resulting in an illuminance of approximately 11,000 lux. The lamps above the left and right edges of the sample stage are directed towards the field of view at approximately 45 degrees.

The camera black reference is determined with the lens cap on. The camera white reference is determined with a Polaroid 803 positive with 15 ms exposure. To account for the warm color illumination bias of floodlamps, the red, green, and blue (RGB) values are offset using the white selection tool in the KS400 software, resulting in corrected RGB values that yield a white image.

Samples are placed on the stage (normal viewing angle) and under 6.4 mm plate glass to minimize topographical effects. Images of the color-bearing graphical portion are acquired at 15 ms exposure.

Image analysis is performed in Matlab (v.6.5.1, release 13, available from Mathworks, Inc. having a place of business located in Natick, Mass., U.S.A.) with the Image Processing Toolbox (v4.0). RGB images are converted to hue, saturation, and value (HSV) space using Matlab's hsv2rgb.m command. Choosing a saturation lower limit of 0.05 (0-1 scale) results in practical detection of all the colored/inked portions of the graphic. The hue, saturation, and value (i.e., luminosity) densiometric distributions are calculated for the detected regions in each image.

### Hunter Color Test

For the present application, hue, luminosity and saturation/vividness can be measured as described in the following paragraphs.

The color scale values of the specimen can be measured using the CIE LAB scale. Measurements are made with a Hunter Color reflectance meter. A complete technical description of the system can be found in an article by R. S. Hunter, ‘photoelectric color difference Meter’, Journal of the Optical Society of America, Vol. 48, pp. 985-95, 1958. Devices specially designed for the measurement of color on the Hunter scales are described in U.S. Pat. No. 3,003,388 to Hunter et al. Additional information can be found at www.hunterlab.com.

Colors can be measured according to an internationally recognized 3-dimensional solid diagram of colors where all colors that are perceived by the human eye are converted into a numerical code. The CIE LAB system is similar to Hunter L, a, b and is based on three dimensions, specifically L*, a*, and b*.

When a color is defined according to this system, L* represents lightness (0=black, 100=white). A shiny white sample will yield an L* value near 100 while a dull black sample will yield an L* value of about 0. a* and b* independently each represent a two color axis, a* representing the axis red/green (+a=red, −a=green), while b* represents the axis yellow/blue (+b=yellow, −b=blue).

A color may be identified by a unique ΔE value (i.e., difference in color from some standard or reference), which is mathematically expressed by the equation:

ΔE*=[(L*_{X.}−L*_{Y})²+(a*_{X.}−a*_{Y})²+(b*_{X}−b*_{Y})²]^{1/2}

where “x” represents the standard or reference sample which may either be a “white” sample or a “colored” sample, e.g., one colored shade may be compared to another colored shade.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent” generally refers to devices which can absorb and contain fluids. For example, personal care absorbent articles refer to devices which are placed against or near the skin to absorb and contain the various fluids discharged from the body.

The term “attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements.

As used herein, the term “color” and its derivatives is intended to mean an individual's perception of the spectral composition of visible light coming from a portion of an object. Color characteristics include hue, saturation and luminosity. Each is a separate color characteristic. Hue is the attribute of a color which allows it to be classified as a given color. Saturation, which is sometimes referred to as vividness, is the intensity of the color. Saturation is the degree of freedom from gray. Luminosity, sometimes referred to as value, is the degree of lightness (paleness) or darkness in a color and is measured from white to black. For example, a blue with white added is a pale color, e.g. baby blue and blue with black added is a dark color, e.g. navy blue. A measurement of hue, saturation and luminosity are described in more detail herein.

The term “complex liquid” describes a liquid generally characterized as being a viscoelastic liquid pertaining primarily to aqueous liquids associated with menstruation.

The term “connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements.

The term “disposable” is used herein to describe articles, such as absorbent articles, that are not intended to be laundered or otherwise restored or reused as an article after a single use.

The terms “elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 50 percent (to 150 percent) of its relaxed length and will recover, upon release of the applied force, at least 40 percent of its elongation.

The term “thermoplastic” describes a material that softens when exposed to heat and which substantially returns to a non-softened condition when cooled to room temperature.

The term “vaginal-health products” includes, but is not limited to, tampons and tampon applicators; pessary and pessary applicators; sanitary napkins, wipes, menstrual pads, menstrual pants, panty liners, panty shields, interlabials and the like.

These terms may be defined with additional language in the remaining portions of the specification.

### DETAILED DESCRIPTION

Vaginal-health products include disposable tampons and pessaries, such as urinary incontinence pessaries. In general, a tampon is a compressed, fibrous, absorbent pledget that is inserted into the vagina. The absorbent pledget has a generally cylindrical bullet shape, and its primary function is to absorb complex fluids, such as menses. The term “digital tampon” refers to an absorbent pledget that a woman inserts directly into her body using her fingers. However, many users are adverse to using their fingers to insert the absorbent pledget. Rather, it can be preferred to insert the absorbent pledget indirectly, such as by utilizing an “applicator tampon.”

In general, a pessary can also be a compressed pledget that is inserted into the vagina, and may be absorbent or non-absorbent. Exemplary disposable pessaries are described in U.S. Pat. No. 6,679,831 to Zunker et al., which is incorporated herein by reference in a manner that is consistent herewith.

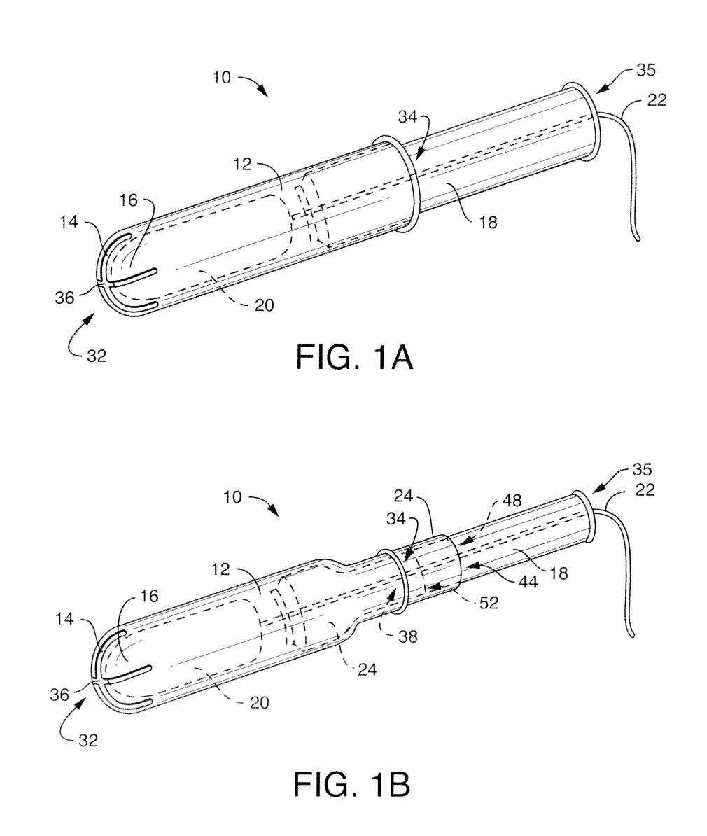

To gain a better understanding of the present invention, attention is directed to FIG. 1A and FIG. 1B for exemplary purposes showing a tampon of the present invention. It is understood that the present invention may be suitable for use with various other vaginal-health products without departing from the scope of the present invention.

FIG. 1A shows one representative version of an applicator tampon. In this version, the applicator 10 typically has two primary components: a barrel portion 12 having slits 14 defining individual petals 16 at the insertion end 32 of the barrel portion 12; and a plunger portion 18 that fits through the opening at the trailing end 34 of the barrel portion 10. An absorbent pledget 20 is contained within the barrel 10. A string 22 is attached to the pledget 20 and protrudes from the trailing end 35 of the applicator 10. After a user inserts the barrel 12 of the applicator 10 into the vagina, the user depresses the plunger 18 so that it pushes the pledget 20 through the opening 36 formed by the individual petals 16. The petals 16 are sufficiently flexible such that they are able to extend outwardly to allow passage of the pledget 20. In this manner the pledget 20 is deployed into the vaginal canal, and the applicator 10 is withdrawn, passing over the string 22 attached to the pledget 20. A user later removes the absorbent pledget 20 by grasping and pulling the string 22.

FIG. 1B shows another representative version of an applicator tampon. In this version, the applicator 10 has three primary components: a barrel portion 12 having slits 14 defining individual petals 16 at the insertion end 32 of the barrel portion 12; an additional component in the form of a telescoping portion 24 that fits within the opening 38 at the trailing end 34 of the barrel portion 12; and a plunger portion 18 that fits through the opening 48 at the trailing end 44 of the telescoping portion 24. This arrangement allows the applicator components to compress inside each other, at least in part, and to remain in a more compact form until use at which time the applicator components are pulled apart and extended to full size. The absorbent pledget 20 is contained within the barrel 12. A string 22, attached to the pledget 20 protrudes from the trailing end 35 of the applicator 10. After a user inserts the barrel 12 of the applicator 10 into the vagina, the user depresses the plunger 18 and the telescoping portion 24 so that they push the pledget 20 through the opening 36 formed by the individual petals 16. The petals 16 are sufficiently flexible such that they are able to extend outwardly to allow passage of the pledget 20. In this manner, the pledget 20 is deployed into the vaginal canal, and the applicator 10 is withdrawn, passing over the string 22 attached to the pledget 20. A user later removes the absorbent pledget 20 by grasping and pulling the string 22.

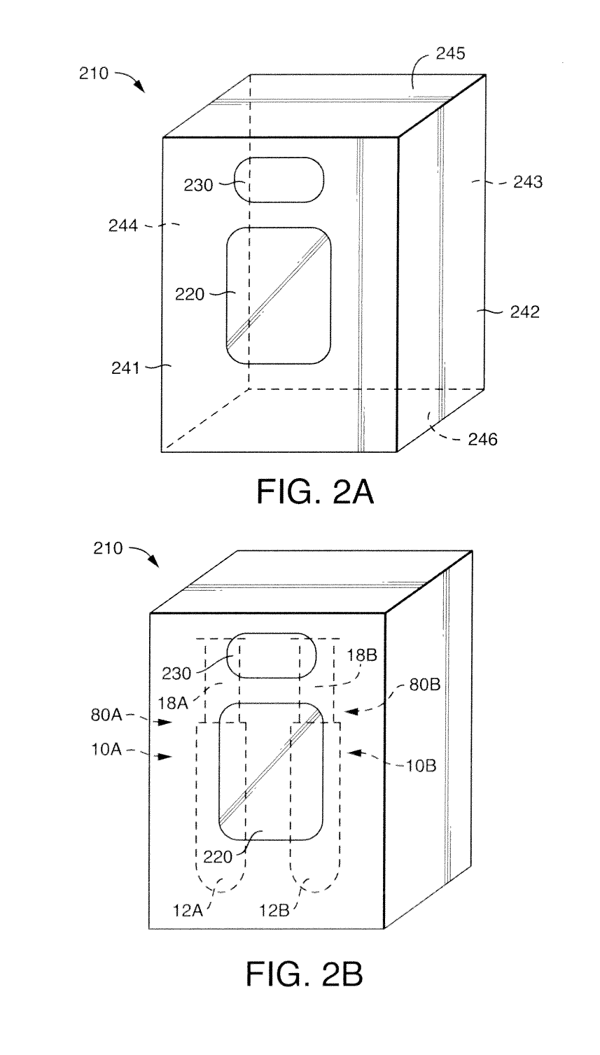

Applicator tampons, like the representative versions shown in FIGS. 1A and 1B, are manufactured by making an absorbent pledget 20, placing the pledget 20 inside an applicator 10, and then placing the applicator 10 in an individual wrapper. Equipment is used to seal the individual wrapper to help protect the applicator tampon until it is used. Selected quantities of individually-wrapped applicator tampons are then placed in a package 210, such as seen in FIGS. 2A and 2B.

For an applicator 10 comprising a barrel portion 12 and a plunger portion 18, at least some part of the plunger portion 18 is placed inside the barrel portion 12. The plunger portion 18 is capable of moving inside the barrel portion 12 (e.g., by sliding, or twisting). Typically, the plunger portion 12, barrel portion 18, or both are fashioned so that the plunger portion 18 cannot be easily separated from the barrel portion 12. For example, the end of the plunger portion 18 inside the barrel portion 12 may comprise, e.g., a rim or protrusion that extends outwardly from the outer surface of the plunger portion. When the plunger portion 18 is pulled so that it slides inside the barrel portion 12, this outwardly extending rim or protrusion cannot readily pass over the barrel trailing end 34, e.g., a rim or protrusion that extends inwardly from the inner surface of the barrel portion 12 at its trailing end 34.

An applicator comprising a barrel portion 12, a telescoping portion 24, and a plunger portion 18 can be constructed in a similar fashion. It may be desirable that some of these portions are partly inside, and can move inside, other portions. Thus, for the representative embodiment depicted in FIG. 1B, the plunger portion 18 is partly inside, and can move inside, the telescoping portion 24, and the telescoping portion 24 is partly inside, and can move inside, the barrel portion 12. If desired, these portions can be fashioned with rims, protrusions, ridges, or other shapes so that the barrel portion 12, telescoping portion 24, and/or plunger portion 18 cannot be readily pulled apart. Furthermore, the trailing end 44 of the telescoping portion 24, and the insertion end 52 of the plunger portion 18 that fits inside of the telescoping portion 24, can be fashioned so that when the plunger 18 is first pulled to fully extend the applicator 10, the plunger 18 snaps into place and locks inside the telescoping portion 24. When the user pushes on the plunger portion 18, both the plunger portion 18 and the telescoping portion 24 to which the plunger portion 18 is now connected (i.e., after the plunger portion 18 is locked into place inside the telescoping portion 24) will move. The combination, then, pushes the absorbent pledget 20 inside the barrel 12 through the insertion end 32 of the barrel portion 12 having the slits 14 and petals 16.

In several embodiments of the invention, some or all of the applicator is constructed of a thermoplastic polymer (referred to herein as “plastic”). During manufacture of the applicator 10, plastic is heated so that it can flow. The heated plastic is introduced into injection-molding equipment where the plastic assumes the form defined by a mold (e.g., the barrel portion 12, plunger portion 18, and optional telescoping portion 24 described above). The components are then connected to one another. When making the components of the applicator 10, particular polymeric material may be used so that a desired component, once formed, is transparent, translucent, or opaque. Furthermore, the components of the applicator 10 may be formed to incorporate a variety of colors to make various aspects of the present invention. Given the nature of vaginal-health products, particular colorants, dyes, pigments, and the like can be selected that are appropriate or desirable for such products.

As referenced above, the barrel 12 contains a pledget 20 that is inserted into the vagina by virtue of a user pressing on the plunger portion 18. When depressed, the plunger portion 18, alone or in combination with other components (e.g., a telescoping portion 24), ejects the pledget 20 through the insertion end 32 of the barrel 12 and into the vaginal cavity. As shown in the figures, this is typically accomplished by pushing the pledget 20 through the insertion end 32 having slits 14 and flexible petals 16 that move outwardly to allow passage of the pledget 20.

The pledget 20 can be loaded into either end 32,34 of the barrel 12 during manufacture of the vaginal-health product. For example, in some aspects, if the applicator 10 is assembled first (e.g., the molded plunger portion 18 is inserted into the molded barrel portion 12 prior to inserting the pledget 20 into the barrel portion 12), then the pledget 20 will typically be loaded into the insertion end 32 of the barrel 12. This can be accomplished by heating the petals 16 and extending them outward, thus allowing the pledget 20 to be inserted into the barrel 12. The heated petals 16 can then be returned to their original position and allowed to cool. Alternatively, the barrel portion 12 may be formed with petals 16 that are in an open position, the pledget 20 is inserted into the insertion end 32, and the petals 16 are then heated, closed and cooled to keep them in a closed position. In other aspects, the pledget 20 may first be placed inside the barrel 12 through the trailing end 34. Then the plunger portion 18 can be inserted into the barrel portion 12.

The above paragraphs give a basic description of some representative versions of vaginal-health products and the manner in which they may be made. Suitable absorbent pledgets and withdrawal string materials and constructions; pessary devices; paperboard and plastic applicator materials and constructions; film and other wrapper materials and constructions used to enclose individual tampon applicators; and film and paperboard packaging materials and constructions are known to those skilled in the art and are not further described unless needed to describe the present invention.

For applicators incorporating two or more components, e.g., a barrel portion 12 and a plunger portion 18, as well as any additional components (such as the exemplary telescoping portion 24 discussed above), the individual components may be made so that they have the same color or different colors, and such colors (whether the same or different) can also be coordinated in some aspects. Furthermore, one or more colors of the applicators or components thereof may be the same as, or different than, colors on the wrapper and/or on the package, wherein the colors can also be coordinated in some aspects.

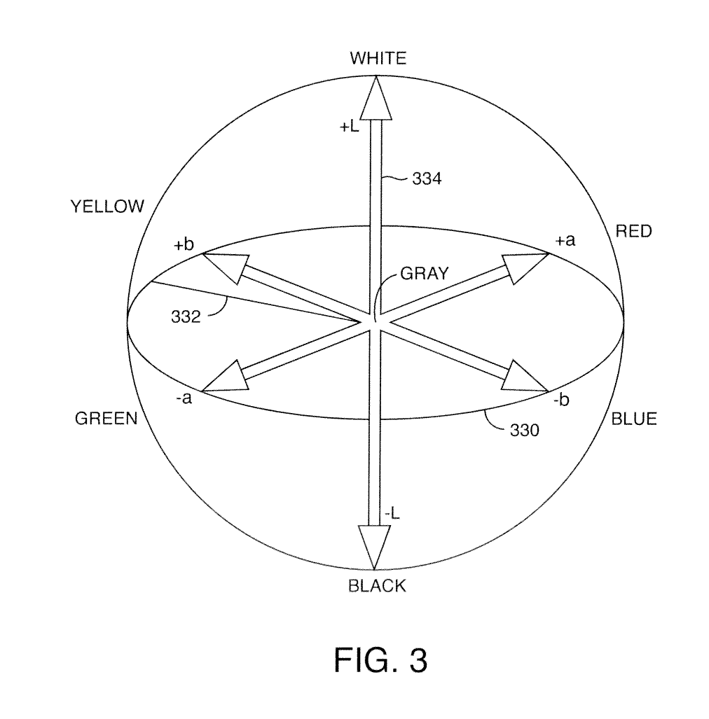

In some aspects of the present invention, the hue, luminosity and saturation/vividness of a color can be determined by the Color Measurement Test, Hunter Color Test, or equivalent. In additional aspects, a color having a particular hue, luminosity and saturation/vividness can be applied to an internationally recognized 3-dimensional solid color chart as would be well-known to those skilled in the art, such as the color chart 310 of FIG. 3, and can be utilized to determine whether a color is “different” and/or “coordinated.” For example, the hue of a particular color can be applied to the color chart 310 along circle 330 having 360 degrees. Thus, in some aspects, two colors are considered to be different herein if they have first and second respective hues that are at least apart from each other when viewed along circle 330, such as at least ±10 degrees, or at least ±30 degrees, or at least ±90 degrees, or at least ±150 degrees or at least ±175 degrees apart when viewed along circle 330. In addition, the value (luminosity) of a color can be applied to the color chart 310 along the z-axis 334 between white and black. Luminosity is measured on a scale from white to black. By way of example only, a luminosity of 100% equates to white while a luminosity of 0% equates to black. Thus, in some aspects, two colors are considered to be different herein if they have a luminosity difference of at least 10% of maximum of the z-axis 334 (using the Polaroid white reference standard), such as at least 15% of maximum or at least 20% of maximum. Further, the saturation/vividness (chroma) of a color can be applied to the color chart 310 along the length of the radius (r) 332. Thus, in some aspects, colors are also considered to be different herein if they have a saturation/vividness difference of at least 20% of maximum of the radius 332, such as at least 25% of maximum or at least 30% of maximum. Accordingly, in some aspects, the present invention is directed to two or more different colored applicators or components thereof in the same package, giving a user the ability to choose one of the available colors (or remaining available color(s) after one or more vaginal-health products comprising these colored applicators have been used) according to a user's particular preference or desire.

As referenced above, in some aspects of the present invention, two colors are considered to be coordinated herein if they have first and second respective hues that are within about ±120 degrees of each other along circle 330 of FIG. 3, such as within ±30 degrees, or within ±15 degrees, or within ±10 degrees, or within ±5 degrees or within ±0.50 degrees of each other. In some aspects, colors are also considered to be coordinated if they have a luminosity difference of less than 5% of maximum of the z-axis 334 (using the Polaroid white reference standard) when applied to the color chart 310 along the z-axis 334 between white and black, such as less than 3% of maximum or less than 1% of maximum. In some aspects, colors are also considered to be coordinated if they have a saturation difference of less than 5.0% of maximum when applied to the color chart 310 along the length of the radius 332, such as less than 3.0% of maximum or less than 2.5% of maximum. Additional information with respect to colors and color charts in general can be found at http://www.applepainter.com, www.hunterlab.com, and http://www.xrite.com/top_munsell.aspx, for example, incorporated herein by reference in a manner that is consistent herewith.

In some aspects, the present invention is directed to vaginal-health products employing an applicator. The applicator is typically made of plastic or paperboard, and contains a pledget to be deployed in a user's vaginal cavity. In some aspects, the pledget may be, for example, an absorbent pledget for use in absorbing fluid such as menses. In other aspects, the pledget may be, for example, a pessary for exerting pressure on a user's urethra to inhibit or stop the flow of urine. Regardless of the specific product form, a given package contains applicators of different colors, with a user being given the choice of which available color to use on a particular occasion.

As referenced above, applicators 10 typically are made up of at least two components or portions: a barrel portion 12; and a plunger portion 18, at least a part of which fits inside the barrel portion 12, and which can slide, twist, or both, inside the barrel portion 12. Alternatively, an applicator 10 may have at least 3 portions: a barrel portion 12; a telescoping portion 24, at least a part of which fits inside the barrel portion 12, and which can slide, twist, or both, inside the barrel portion 12; and a plunger portion 18, at least a part of which fits inside the telescoping portion 24, and which can slide, twist, or both inside the telescoping portion 24. Other variations are also possible. For example, the barrel portion 12 can be fitted with an elastomeric grip (not shown) at that location where a user typically grips the applicator 10 when deploying the pledget 20 inside the applicator 10.

Regardless of the specific construction of the applicator 10, at least a portion of the applicator 10, typically the barrel 12, is made so that, in a given package 210 of vaginal-health products comprising applicators 12, a user can choose a colored applicator from two or more different colored applicators.

In some aspects, all of the components of a given applicator 10 can be the same color. However, two or more components of an individual applicator need not match. For example, in some aspects, for a two-component applicator, the color of the barrel portion 12 of the applicator can be varied, with a user picking an applicator having a barrel color that the user wishes to use at that particular time. In some aspects, the plunger portion 18, however, could be the same color for all of the applicators.

In some aspects, the color of the plunger portion 18 might be used to convey information. For example, directions on or in the package 210, whether given by text, icons, or some other form of communication, could communicate that a user, when ready to deploy the pledget 20 inside the vaginal-health product (e.g., an absorbent pledget in an applicator tampon), should depress the colored plunger, while highlighting the specific color of the plunger.

In other aspects, this scheme could be reversed: i.e., the barrel 12 could be the same color for all the applicators, and the plunger 18 could be of different colors. It is understood that the component of the applicator that is the same color for all applicators could be used to convey information about other aspects of the product (e.g., signify absorbency level).

In some aspects, different colored barrels could be assembled with different colored plungers. This could be done so that there are random combinations of colored barrels and colored plungers. In some aspects, the combinations of colored barrels and colored plungers could reflect a decorative theme. For example, each of the different colored barrels could be a darker shade of a particular color. For example, the barrels could be one of four different colors: a dark green; a dark blue; a dark yellow; and a dark pink. The corresponding plungers could be selected so that they are the same color as each corresponding barrel, but of a lighter shade. Thus, a light green plunger is matched with a dark green barrel; a light blue plunger is matched with a dark blue barrel; a light yellow plunger is matched with a dark yellow barrel; and a light pink plunger is matched with a dark pink barrel. Of course other combinations of shades are possible. In other aspects, the combinations of dark and light shades mentioned above could be reversed (e.g., a dark green plunger matched with a light green barrel, etc.).

The components of the applicator 10 need not be opaque. For example, the components can be transparent, transparent and tinted with color, translucent (with or without color), or possess other combinations of light transmittance and color attributes. Thus, a package 210 might contain a vaginal-health product with applicators 10 in which the barrel portions 12 comprise different colors. The plungers 18 however, could be clear without any tint or color. In other examples, the plungers 18 could be clear, or substantially clear but tinted. If the plungers 18 are colored in some way, the color of the plunger 18 could be different from that of the barrel 12, or the same or similar. As noted above, the plunger 18 could have a different hue or shade from the barrel 12. Also, as noted above, the barrels 12 could be of different colors, but the plunger 18 might be made of one color tint, possibly with the color conveying information about the product or its use.

The preceding paragraphs emphasize an applicator 10 having two primary components: e.g., a barrel portion 12 and a plunger portion 18. The present invention can also be suitable for applicators 10 having more than two primary components, which accordingly provides increased options for mixing and matching colors. Thus, for example, an applicator 10 may comprise 3 primary components: a barrel portion 12, a telescoping portion 24 that fits inside the barrel portion 12, and a plunger portion 18 that fits inside the telescoping portion 24.

In one exemplary version of the invention, the barrel 12 and the plunger 18 are the same or similar colors (e.g., the barrel and the plunger may be different shades or hues of the same color). The inter-connecting telescoping portion 24 can be the same or similar color; transparent, translucent, or opaque; tinted; or another color. Other variations are of course possible. As stated above, the color scheme of the applicator 10 may be such that the color, or colors, of the different components of the applicator 10 do not signify a functional characteristic, use feature (possibly tied to directions for use, as in the example discussed above), or the like. Alternatively, so long as the user is able to choose applicators comprising at least one component that has different colors (e.g., a package 210 in which the barrel 12 of the applicators 10 is green, blue, yellow, or pink), then another component of the applicator may be one color that signifies a functional characteristic or use feature of the product.

In some aspects, a component of the applicator 10 may incorporate more than one color (e.g., swirl patterns comprising two or more colors).

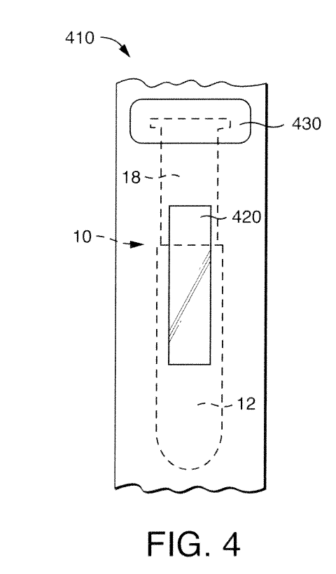

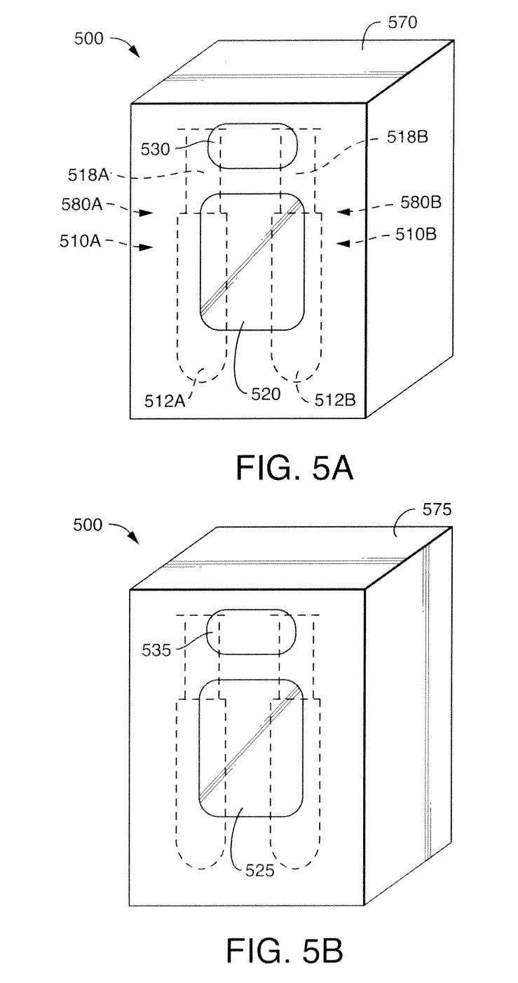

Typically, vaginal-health products comprising applicators 10 that contain pledgets 20, such as absorbent pledgets or pessary devices, are individually wrapped in a wrapper 410 (FIG. 4) to protect the product until it is used. In some versions of the invention, some portion of the wrapping material may be sufficiently transparent so that the color of the applicator 10 contained within the wrapper 410 is visible. If one portion of the applicator 10 is made with different colors from which a user can choose, and another component of the applicator 10 is a single color (e.g., to signify a functional characteristic or use feature), then the transparent portion 420 of the wrapper 410 may be of a size, or location, such that the different-colored portion is visible, so that the user can pick that color of interest for a particular use. Alternatively, the wrapper 410 may comprise indicia 430 allowing a user to pick a specific colored applicator 10 from those available in a package 210. Thus the wrapper 410 may comprise the same color as at least one of the colored components of the applicator 10 contained in the wrapper 410 (e.g., the wrapper may be yellow, or have a yellow ornamental design, with the yellow color matching, or being coordinated with, the yellow color of the barrel portion 12 of the applicator 10).

As stated above, the present invention is directed to vaginal-health products comprising applicators 10 that contain a pledget 20 such as an absorbent pledget or pessary device. In a given package 210, these pledgets 20 are typically the same. So, for example, each of the different colored applicators in a given package 210 contains the same absorbent pledget (e.g., an absorbent pledget of a given absorbency level, such as “Regular”). In other aspects, each of the different colored applicators in a given package contains the same pessary device (i.e., a pessary device of a given design and size).

Packages 210 containing individually wrapped, vaginal-health products comprising applicators 10 can have a viewable surface area. In some aspects, to further support that the vaginal-care products of the present invention promote openness and are far from the taboos that historically are linked to the vaginal-health category, at least 30% of the viewable surface area of the package is black. If the package has the general shape of a rectangular parallelepiped such as illustrated in FIG. 2A (i.e., four rectangular side panels 241,242,243,244, a rectangular top panel 245, and a rectangular bottom panel 246), then “viewable surface area” means the sum of the six areas corresponding to each of the six viewable rectangles or panels 241,242,243,244,245,246 that make up the rectangular parallelepiped. Having multiple colors in a package is starkly different from packages that have been offered historically in the vaginal-health category. Accordingly, other versions of the invention have even higher percentages of the viewable surface area as black, such as 50%, 75%, or even 90% or more.

Furthermore, packages containing individually wrapped, vaginal-health products comprising applicators 10 can include windows through which one or more of the wrapped products are viewable. If these wrappers 410 are different colors, as would be the case if the wrappers comprise an indicia 430, such as a colored indicia matching a color of the applicator 10 contained inside the wrapper 410, then one or more of these colored wrappers would be viewable by virtue of the window 420. In some aspects, to further support that the vaginal-health products of the present invention break taboos, and break with the historic link between feminine care and a medical condition, the package 210 can comprise both a window 220 and a viewable surface area that is at least 30% black, desirably the package viewable surface area near the window 220. This stark contrast between a package 210 comprising the color black, and multi-colored wrappers 410 viewable through a window 220, helps support this new, modern, open innovation.

In some aspects, when the individually wrapped, vaginal health products are placed in the package 210, they may be placed randomly, or according to some pre-selected arrangement. For example, if each of the wrappers 410 comprises a color that is the same as the color of an applicator 10 contained inside the wrapper 410, then these different colored applicators may be inserted into the package 210 in some pre-selected order (e.g., first, a wrapper comprising red that contains an applicator comprising red; second, a wrapper comprising yellow that contains an applicator comprising yellow, third, a wrapper comprising blue that contains an applicator comprising blue; with this sequence then repeating red, yellow, blue). Alternatively, the wrappers 410 may be inserted randomly such that one or more wrappers of the same color may, due to chance, be placed next to each other inside the package 210.

Some aspects of the invention are directed to arrays 500 of vaginal-health products. In one particular aspect, the array 500 comprises a first package 570 and second package 575, each containing vaginal-health products. The first package 570 comprises a first vaginal-health product 580A and a second vaginal-health product 580B. The first vaginal-health product 580A comprises a first applicator 510A, the first applicator having a first barrel portion 512A and a first plunger portion 518A. The first barrel portion 512A comprises a first barrel color. The first vaginal-health product 580A also comprises a first pledget (not shown) that is at least partly contained in the first barrel portion 512A. The second vaginal-health product 580B comprises a second applicator 510B, the second applicator having a second barrel portion 512B and a second plunger portion 518B. The second barrel portion 512B comprises a second barrel color. The second vaginal-health product 580B also comprises a second pledget (not shown) that is at least partly contained in the second barrel portion 512B. The first barrel color is different from the second barrel color, and the first pledget is the same as the second pledget. The array also comprises a second package 575. The second package 575 contains vaginal-health products comprising applicators, with the applicators in the second package each having the same appearance. This type of array can reflect a brand comprising two different sub-brands. The first sub-brand corresponds to the first package 570, and the second sub-brand corresponds to the second package 575.

In other aspects, an array similar to that described in the preceding paragraph includes the first package 570 having a first viewable surface area, and at least 30% of the first viewable surface area is black. The second package 575 has a second viewable surface area, and at least 30% of the second viewable surface area is white. The packages may be made of plastic film, paperboard, or some combination of these. In one version of the invention, the first package 570 is composed of paperboard, and the second package 575 is composed of plastic film. One or both of the packages may also incorporate a window 520,525 through which the contents of the packages are viewable. In other aspects, the first package 570 comprises a window 520 through which the contents of the first package 570 are viewable, and the second package 575 does not comprise a window. Again, these arrays may reflect a brand comprising two different sub-brands. The first sub-brand corresponds to the first package 570, and the second sub-brand corresponds to the second package 575.

In some aspects, the package 210 has features similar to those described generally in the preceding paragraphs. For example, in one aspect, the invention is a package 210 containing vaginal-health products. The package includes a first vaginal-health product 80A and a second vaginal-health product 80B. The first vaginal-health product 80A has a first applicator 10A. The first applicator 10A has both a first barrel portion 12A and a first plunger portion 18A, with the first barrel portion 12A having a first barrel color. A first pledget (not shown), such as an absorbent pledget or a pessary device, is at least partly contained in the first barrel portion 12A. The package also includes a second vaginal-health product 80B. The second vaginal-health product 80B has a second applicator 10B. The second applicator 10B has both a second barrel portion 12B and a second plunger portion 18B, with the second barrel portion 18B having a second barrel color. A second pledget (not shown) is at least partly contained in the second barrel portion 12B. The first barrel color is different from the second barrel color, and the first pledget is the same as the second pledget. The package can also include indicia 230 relating the first barrel color to a state of being of the user. Furthermore, the package 210 may comprise indicia 230 pertaining to a user's ability to choose different colors.

In some aspects, an array 500 of vaginal-health products comprises a first package 570 having indicia 530 of a first sub-brand, the first package 570 containing different colored applicators; and a second package 575 having indicia 535 of a second sub-brand, the second package 575 containing applicators of a single color. In some particular aspects, the first package 570 in the array 500 comprises a transparent portion through which the individual wrappers containing the different colored tampon applicators may be seen. As referenced above, to facilitate a user's choice of applicator color, some portion of the wrapper may be substantially the same as the applicator color contained therein.

In some aspects, an array 500 of vaginal-health products comprises a first package 570 containing different colored applicators, each of which is individually wrapped, with each individual wrapper having some portion that is substantially the same color as the applicator color contained therein; and a second package 575 containing pads, each of which is individually wrapped, with each individual wrapper having a different color.

It should be understood that “different colored applicators” does not require that each and every applicator in the package be different from the others. This term, and other similar terms, encompasses sets of applicators in which a user may choose from two, three, four, or more different colors. Thus, for example, the term encompasses a set of two applicator vaginal-care products, with each of the two corresponding applicators being differently colored from the other. In another example, the term encompasses a set of three applicator vaginal-care products, with one of the corresponding applicators having a substantially blue color, and the remaining two applicators having a substantially green color. In another example, the term encompasses a set of eight applicator vaginal-care products, with two of the corresponding applicators having a blue color; two having a green color; two having a yellow color; and two having a red color. Many other versions are of course possible, with a desirable feature being that a user can pick one color from at least two or more available colors (of course the choice of colors will be reduced, eventually, to one as the user employs and discards the products contained in a package).

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the desirable embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. A package containing vaginal-health products, the package comprising:
a first vaginal-health product comprising
a first applicator, the first applicator comprising a first barrel portion and a first plunger portion, wherein the first barrel portion comprises a first barrel color;
and a first pledget at least partly contained in the first barrel portion; and
a second vaginal-health product comprising
a second applicator, the second applicator comprising a second barrel portion and a second plunger portion, wherein the second barrel portion comprises a second barrel color;
and a second pledget at least partly contained in the second barrel portion;
wherein the first barrel color is different from the second barrel color, and
wherein the first pledget is the same as the second pledget.

2. The package of claim 1 wherein the first plunger portion comprises a first plunger color, the second plunger portion comprises a second plunger color, and the first plunger color is the same as the second plunger color.

3. The package of claim 2 on which are disposed instructions on use of the vaginal-health product that reference the color of the plunger.

4. The package of claim 1 wherein the first plunger portion comprises a first plunger color that is substantially the same as the first barrel color, and the second plunger portion comprises a second plunger color that is substantially the same as the second barrel color.

5. The package of claim 1 wherein the first plunger portion comprises a first plunger color that is different from the first barrel color, and the second plunger portion comprises a second plunger color that is different from the second barrel color.

6. The package of claim 1 wherein the first plunger portion comprises a first plunger color that is a different shade of the first barrel color, and the second plunger portion comprises a second plunger color that is a different shade of the second barrel color.

7. The package of claim 1 wherein the first vaginal-health product is contained in a first wrapper comprising a first wrapper color that is substantially the same as the first barrel color, and the second vaginal-health product is contained in a second wrapper comprising a second wrapper color that is substantially the same as the second barrel color to provide wrapped vaginal-health products.

8. The package of claim 1, wherein the arrangement of vaginal-health products inside the package comprise a pattern.

9. The package of claim 1 wherein the first vaginal-health product is contained in a first wrapper comprising a window through which a portion of the first vaginal-health product is viewable, and the second vaginal-health product is contained in a second wrapper comprising a window through which a portion of the second vaginal-health product is viewable.

10. The package of claim 1 wherein the first pledget and the second pledget are each an absorbent pledget having the same absorbency level.

11. The package of claim 10 wherein the first vaginal-health product is contained in a first wrapper comprising a first wrapper color that is substantially the same as the first barrel color; wherein the second vaginal-health product is contained in a second wrapper comprising a second wrapper color that is substantially the same as the second barrel color; wherein the first wrapper further comprises a first indicia of the absorbency level; the first indicia being unrelated to the first barrel color; and wherein the second wrapper further comprises a second indicia of the same absorbency level, the second indicia being unrelated to the second barrel color.

12. The package of claim 1, wherein the package has a viewable surface area, wherein at least 30% of the viewable surface area is black.

13. The package of claim 12 further comprising a window through which a portion of the contents of the package is viewable.

14. The package of claim 1 wherein the package further comprises indicia relating at least the first barrel color to a state of being of a user.

15. The package of claim 1 wherein the package further comprises indicia relating to a user's ability to choose different colors.

16. An array of packages of vaginal-health products, the array comprising:
a first package containing vaginal-health products, the first package comprising:
a first vaginal-health product comprising
a first applicator, the first applicator comprising a first barrel portion and a first plunger portion, wherein the first barrel portion comprises a first barrel color;
and a first pledget at least partly contained in the first barrel portion; and
a second vaginal-health product comprising
a second applicator, the second applicator comprising a second barrel portion and a second plunger portion, wherein the second barrel portion comprises a second barrel color;
and a second pledget at least partly contained in the second barrel portion;
wherein the first barrel color is different from the second barrel color, and wherein the first pledget is substantially the same as the second pledget; and
a second package containing vaginal-health products comprising applicators, wherein the applicators in the second package all have the same appearance; and
wherein the first package and the second package have indicia of the same brand.

17. The array of claim 16, wherein the first package has a first viewable surface area, and at least 30% of the first viewable surface area is black; and wherein the second package has a second viewable surface area, and wherein at least 30% of the second viewable surface area is white.

18. The array of claim 16, wherein the first package is composed of paperboard, and wherein the second package is composed of plastic film.

19. The array of claim 16, wherein at least the first package comprises a window.

20. A package containing vaginal-health products, the package comprising:
a first vaginal-health product comprising a first applicator having a first applicator color, wherein the first applicator is disposed within a first wrapper having a first wrapper color;
a second vaginal-health product comprising a second applicator having a second applicator color, wherein the second applicator is disposed within a second wrapper having a second wrapper color;
wherein the the first applicator color is the same as the second applicator color; and
wherein the first wrapper color is different than the second wrapper color.

21. The package of claim 20, wherein the first wrapper comprises a first indicia having a first indicia color and the second wrapper comprises a second indicia having a second indicia color; wherein the first indicia color and the second indicia color are unrelated to the first applicator color and the second applicator color; wherein the first indicia color is the same as the second indicia color; wherein the first indicia color and the second indicia color indicate the absorbency level of the first vaginal-health product and the second vaginal-health product; and wherein the first vaginal-health product and the second vaginal-health product have the same absorbency level.

22. The package of claim 20 further comprising a window through which a portion of the contents of the package are viewable.

23. The package of claim 20, wherein the package has a viewable surface area, wherein at least 30% of the viewable surface area is black.
